(19) United States
(12) Patent Application Publication
(10) Pub. No.: US 2005/0060022 A1
(43) Pub. Date: Mar. 17, 2005

(54) Polymer stent

(76) Inventors: Jeffrey C. Felt, Excelsior, MN (US); Alexander Arsenyen, Eagan, MN (US); Mark A. Rydell, Golden Valley, MN (US)

Correspondence Address: BECK AND TYSVER, 2900 THOMAS AVENUE SOUTH, SUITE 100, MINNEAPOLIS, MN, 55416, US

(21) Appl. No.: 10/850,501

(22) Filed: Nov. 26, 2004

Related U.S. Application Data
(60) Provisional application No. 60/472,125, filed on May 21, 2003.

(51) Int. Cl. A61F 2/06

(52) U.S. Cl. 623/1.15; 623/1.42; 623/1.44

(57) ABSTRACT

A polymer intravascular stent that is resorable over a period of time containing a drug, which is delivered to the vascular wall during the absorption of the polymer material. A gradient of drug concentration may be established in the polymer.

### CROSS REFERENCE

The present case is the utility application based upon US Provisional Application 60/472,125 filed May 21, 2003 entitled “Drug Eluting Polyurethane Stent” which is incorporated by reference herein in its entirety.

### FIELD OF THE INVENTION

The present invention relates generally to a medical device, and more particularly to a stent with a polymer coating or polymer structure containing a drug to. prevent restenosis.

### BACKGROUND OF THE INVENTION

Stents are widely used for treating coronary vessel disease as well as for propping open other lumens within the body. The earliest form of stent construction involved a “bare metal” wire mesh structure. Later some versions of the device were made from a material exhibiting a “shape memory” attribute. Devices of this type can “self deploy”. Other versions of the device were deployed through the use of a balloon expansion device that expanded the stent into contact with the wall of the vessel or lumen.

Bare metal stents in the vasculature have a long history and the cascade of injury and the associated healing process that accompanies recovery from stent placement has been extensively studied. This healing response encapsulates the stent with neointimal tissue growth. It is widely known that in some instances the healing response can ultimately lead to an occlusion of the vessel, which is referred to as restenosis.

More recently drug-eluting stents have been introduced. These devices deliver a drug to the site of vessel injury and they have been shown to reduce the restenosis rate. It is believed that drugs operate by modulating the healing response. Several classes of drugs including the drugs known as Rapamycin and Taxol and their derivatives are candidates for inclusion into a stent product. Although these drug-eluting stents have been shown to be both safe and effective, once the drug is delivered or “fully gone” the polymer coating that is exposed may not be benign in the vessel. There have been some instances of “late” thrombosis, which appears to be triggered by an inflammatory reaction initiated by the polymer coat used to store and release the drug.

Polyurethane has been widely used in implantable medical devices, and it is clear that there are wide variations in the mechanical properties and biocompatibility of polyurethane based in part on formulation and in part on processing details. In general polyurethanes have been regarded as unsuitable for stent products due to the inflammatory reaction usually provoked by the polymer in a blood vessel. In spite of this history at least one organization CardioTech International of Woburn, Mass. has experimented with stents of polyurethane materials.

### SUMMARY OF THE INVENTION

The present application discloses a polymer stent as well as a polymer coated stent. Both the polymer formulation and a processing methodology are disclosed. The polyurethane described appears to be “more” biocompatible than other formulations. This improved material and its processing may be used to create stents or stent coatings that have superior performance in the body.

Although the polyurethane can be formulated from any of several diisocyanates and from any of several polyols the preferred formulation encompasses aromatic or aliphatic di-and (or) polyisocyanatess such as isomers of phenylene diisocyanate (p-PDI, m-PDI), isomers methylene diphenyl diisocyanate(4,4′ MDI,2,4′MDI,2,2MDI), hydrogenated MDI(H12MDI), toluene diisocyanates(TDI), hexamethylene diisocyanate(HDI) and combinations thereof. Chain extenders can be added to formulate the necessary levels of mechanical properties at body temperature and in the hydrated state. Radiopacity of the bulk material can be improved with the addition chain extenders with bromine or iodine atoms and isocyanate reactive groups.

Some examples of the soft segment forming components are: polytetramethyleneoxide (PTMO) MW 250-2800 Daltons, aliphatic polycarbonate based olygomers MW 600-5000 Daltons, hydroxyl-terminated or amino-terminated olygomers with linear or branched aliphatic backbone such as polyisoprene, polybutadiene, polyisobutylene, carbinol terminated polydimethylsiloxanes (PDMS) or combination of the above-mentioned components.

In the preferred embodiment 4,4 MDI would be used with PTMO polyols of molecular weight 1000 to 2000 Daltons. The flexion modulus will be adjusted by changing hard segment/soft segment ratios and by using cross linking when needed. Softer, more flexible material will be formulated in this way for the smaller more tortuous vessel.

### BRIEF DESCRIPTION OF THE DRAWINGS

Throughout the various drawing like reference numerals indicate identical or equivalent structure wherein:

FIG. 1 is a stent in the compressed state;

FIG. 2 is a stent in the expanded state;

FIG. 3 is a cross section of a monolithic stent;

FIG. 4 is a cross section of a coated or encapsulated stent;

FIG. 6 is a graph depicting the performance of the polymer;

FIG. 7 is a graph depicting the performance of the polymer; and,

FIG. 8 is a graph depicting the performance of the polymer.

### DETAILED DESCRIPTION

Stent Construction

FIG. 1 shows an interluminal stent 10 of the well known “Palmaz” type. In general stents of this type are made from a lattice or mesh form body 12 made from metal. Nitinol and stainless steel are both used for stents of this type. Typically the stent is laser cut and then coated with a polymer for controlled elution of a drug. The drug loaded stent is then crimped onto a balloon delivery system with an overall diameter of “d” and sterilized. During delivery, the stent 10 is expanded into a delivered state seen in FIG. 2. In this condition the stent is expanded to a diameter “d”. Some portions of the stent remain fairly invariant in length such as strut element 14, however other elements such as strut 16 are plastically deformed. The strut structures form a series of interlaced apertures. It is expected that the entire stent will be coated with the polymer and that mechanical properties of the polymer will accommodate flexure and plastic deformation without delaminating or other failures. This may require substantial “soft” segments in formulation.

FIG. 3 shows a cross of section of a “monolithic” stent molded from a polyurethane polymer 20 with mechanical properties tailored to allow for plastic deformation of element 16 or shape memory recovery of the expanded shape. Detail design of these struts will require additional study of the polymer and its processing. Monolithic stents made in this fashion will have the ability to contain and elute drugs over a longer time than a conventional coated stent. This attribute is a consequence of the larger amount of polymer in the device.

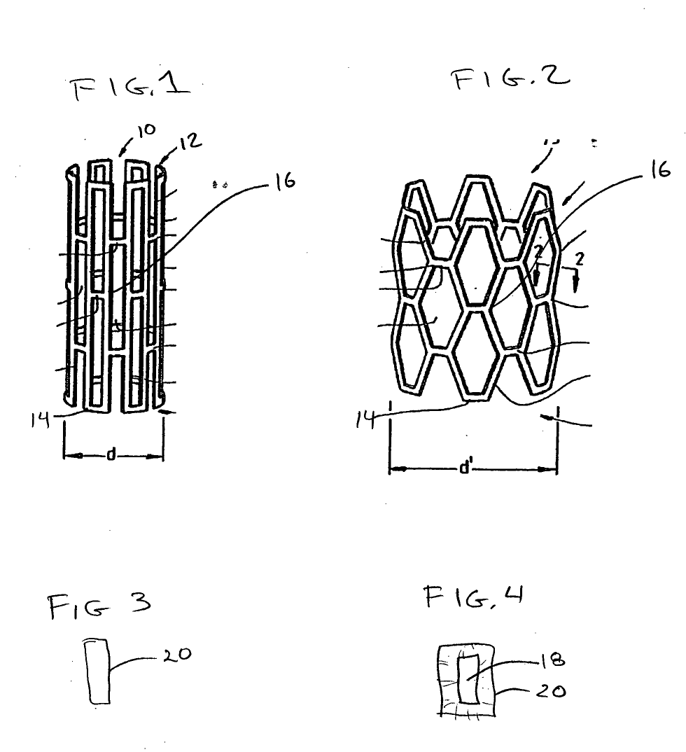

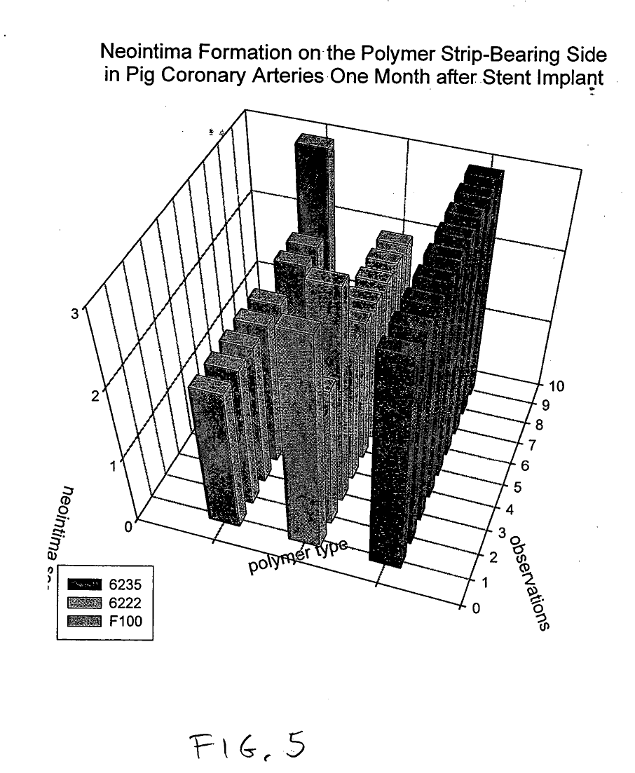

FIG. 4 shows a cross section a metal body 18 with a polymer coat or polymer encapsulation 20 layer. In general the polymer coat may be located only on selected sections of the stent or it may overlay the entire stent. In general the depth of the coating is tailored for mechanical and or pharmacokinetic purposes and results. Once again additional study will be required to perfect a specific design. It may be useful to weave a fabrics from the polymer and to wrap the fabric around the stent. In general the fabric embodiment will increase the surface are that can be used to delivery drug.

Polymer Formulation and Processing

The preferred polymer is a strong, flexible cross-linked polyurethane that can be low pressure injected molded to a final shape. This bulk polymer can be loaded with large molecular weight biologically active molecules that will elute over time. The elution kinetics depend on the operating environment and the specific behavior of the selected drug. The general principle is that the drug is incorporated in the quaternary structure of the aromatic based polyurethane. The depth of the “coat” allows for the creation of a concentration gradient that allows drug to migrate toward the surface providing a longer term continuous delivery of drug

The biomaterial used in this invention preferably includes polyurethane components that are reacted ex vivo to form a polyurethane (“PU”). The formed PU, in turn, includes both hard and soft segments. The hard segments are typically comprised of stiffer oligourethane units formed from diisocyanate and chain extender, while the soft segments are typically comprised of one or more flexible polyol units. These two types of segments will generally phase separate to form hard and soft segment domains, since they tend to be incompatible with one another. Those skilled in the relevant art, given the present teaching, will appreciate the manner in which the relative amounts of the hard and soft segments in the formed polyurethane, as well as the degree of phase segregation, can have a significant impact on the final physical and mechanical properties of the polymer. Those skilled in the art will, in turn, appreciate the manner in which such polymer compositions can be manipulated to produce cured and curing polymers with desired combination of properties within the scope of this invention.

The hard segments of the polymer can be formed by a reaction between the diisocyanate or multifunctional isocyanate and chain extender. Some examples of suitable isocyanates for preparation of the hard segment of this invention include aromatic diisocyanates and their polymeric form or mixtures of isomers or combinations thereof, such as toluene diisocyanates, naphthalene diisocyanates, phenylene diisocyanates, xylylene diisocyanates, and diphenylmethane diisocyanates, and other aromatic polyisocyanates known in the art. Other examples of suitable polyisocyanates for preparation of the hard segment of this invention include aliphatic and cycloaliphatic isocyanates and their polymers or mixtures or combinations thereof, such as cyclohexane diisocyanates, cyclohexyl-bis methylene diisocyanates, isophorone diisocyanates and hexamethylene diisocyanates and other aliphatic polyisocyanates. Combinations of aromatic and aliphatic or arylakyl diisocyanates can also be used. The isocyanate component can be provided in any suitable form, examples of which include 2,4′-diphenylmethane diisocyanate, 4,4′-diphenylmethane diisocyanate, and mixtures or combinations of these isomers, optionally together with small quantities of 2,2′-diphenylmethane diisocyanate (typical of commercially available diphenylmethane diisocyanates). Other examples include aromatic polyisocyanates and their mixtures or combinations, such as are derived from phosgenation of the condensation product of aniline and formaldehyde. It is suitable to use an isocyanate that has low volatility, such as diphenylmethane diisocyanate, rather than more volatile materials such as toluene diisocyanate. An example of a particularly suitable isocyanate component is the 4,4′-diphenylmethane diisocyanate (“MDI”). Alternatively, it can be provided in liquid form as a combination of 2,2′-, 2,4′- and 4,4′-isomers of MDI. In a preferred embodiment, the isocyanate is MDI and even more preferably 4,4′-diphenylmethane diisocyanate.

A number of different anti-metabolities or other drugs can be used and incorporated into the polyurethane stent including the macrocyclic lactones of the sirolimus family, as well as the well established Taxol and Rapamycin drugs. Other drugs such as Hydroxychloroquine and Sulfsalazine along with anti-inflammatory drugs may be used. It is also anticipated that epithelial growth factors may be useful with the polymer. For example two drugs can be selectively loaded into the stent structure and the surface facing the lumen may have a different drug than the surface in contact with the vessel wall.

Experimental Data

To model the behavior of the polymer stent in vivo, polymer was formulated and processed to form short segments of material called “strips”. These strip were cut from a larger section of polyurethane injection molded to form a knee implant. The mold temperature was very high for polyurethane approximately (185 degrees C.). The implant was removed from the mold after only a few minutes (3-5 Minutes) and then washed in a 50% methanol/water solvent for 72 hours at 70C.

These strips were then implanted in pig arteries and allowed to mature for 30 days. The molded polymer was trapped against the wall of the artery by a bare metal stent. After sacrifice, histology was performed and the preferred polymer (F100) was compared with other polyurethanes (6222 and 6235) as controls and the results showed limited fibrotic growth which shows improved biocompatibility with the artery.

General Preparation of F100

In general a polyurethane of the present invention is prepared in the following manner. An amount (51.66 grams, 44.99 weight percent) of diphenylmethane 4,4_-diisocyanate (also known as MDI, available from Bayer under the tradename Mondur M) was added to 27.2 grams (23.69 weight percent) of polytetramethyleneetherglycol 1000 (as available from E.I. du Pont de Nemours and Co. under the tradename Terathane 1000), 20.64 grams (17.98 weight percent) of polytetramethyleneetherglycol 2000 (as available from E.I. du Pont de Nemours and Co. under the tradename Terathane 2000), 14.57 grams (12.69 weight percent) of 1,4-butanediol (as available from Sigma Aldrich Corp.), 0.25 grams (0.22 weight percent) of 2-ethyl-2-hydroxymethyl)-1,3-propanediol (also known as trimethylolpropane, as available from Sigma Aldrich Corp.), 0.50 grams (0.43 weight percent) of pentaerythritol tetrakis (3-(3,5-di-tert-butyl-4-hydroxyphenyl) proprionate (available from Ciba Specialty Chemicals, Inc. as Irganox 1010), and 0.0011 grams (0.0010 weight percent) of bis-(dodecylthio)-dimethylstannane (available from Crompton Corp. as Fomrez catalyst UL-22) to make 114.83 grams of polyurethane within the scope of the present invention.

Polymer mixing equipment was cleaned and dried in a vacuum oven using normal laboratory techniques. Polymer materials were weighted and placed into a mixer, and mixed under vacuum. After additional de-gassing under vacuum, the polymer was injected into a mould at high temperature (185C) where it remained for a short time of about 3 minutes. After molding the parts were annealed at 120degrees C. in an aluminum pouch for about 20 hours. The aluminum pouch is intended to exclude air and humidity form the material during the annealing process. Unless otherwise indicated, all parts are by weight, based on the weight of the final composition.

Preparation of Polymeric Biomaterials (F100 and Control)

Biomaterials of the present invention were provided in the form of polyurethane compositions having different compositions set forth in TABLE I below.

TABLE I
Compositions of Various Suitable Biomaterials

| | Com- | Com- |
|---|---|---|
| | pound A | pound B |
| Ingredient | Weight % | Weight % |
| Isocyanate-diphenylmethane 4,4'-diisocyanate | 44.99 | 36.79 |
| (also known as MDI, available from Bayer under | | |
| the tradename Mondur M) | | |
| Polyol-polytetramethyleneetherglycol 1000 (as | 23.69 | — |
| available from E.I. du Pont de Nemours and Co. | | |
| under the tradename Terathane 1000) | | |
| Polyol- | 17.98 | 22.92 |
| polytetramethyleneetherglycol | | |
| 2000 (as available from E.I. du Pont de Nemours | | |
| and Co. under the tradename Terathane 2000) | | |
| Polyol-Polytetrahydrofuran, molecular weight of | — | 23.47 |
| 650, also known as PTHF 650 (also known as | | |
| PTMO 650, as available from E.I. du Pont de | | |
| Nemours and Co.) | | |
| Chain extender- | 12.69 | 8.29 |
| 1,4-butanediol (as available from Sigma Aldrich | | |
| Corp.) | | |
| Crosslinker 2-ethyl-2-(hydroxymethyl)-1,3- | 0.22 | — |
| propanediol (also known as trimethylolpropane, | | |
| as available from Sigma Aldrich Corp.) | | |
| Catalyst-Bis-(dodecylthio)-dimethylstannane | 0.0010 | — |
| (available from Crompton Corp. as Fomrez | | |
| catalyst UL-22) | | |
| Catalyst-tri-ethylene diamine (also known as | 0.04 | — |
| Dabco as available from Air Products) | | |
| Catalyst- Cotin 222 (available from CasChem, | — | 0.02 |
| Inc.) | | |
| Antioxidant- Pentaerythritol Tetrakis (3-(3,5-di- | 0.43 | — |
| tert-buyl-4-hydroxyphenyl)proprionate (available | | |
| from Ciba Specialty Chemicals, Inc. as Irganox | | |
| 1010) | | |
| Antioxidant- Vitamin E (available from Ciba | — | 0.10 |
| Specialty Chemicals, Inc.) | | |
| Hydrophobic additive - | — | 8.36 |
| hydroxyl-terminated Polybutadiene (Wq. Wt. = | | |
| 550)(also known as Poly BD 20LM as available | | |
| from Sartomer) | | |
| Dye Green GLS (available from Clariant Corp.) | — | 0.01 |
| Total | 100.00 | 100.00 |

As an example, a polymeric biomaterial having the composition of Compound A (F 100) may be produced by making a prepolymer of all the ingredients listed above except 1,4-butanediol chain extender and Bis-(dodecylthio)-dimethylstannane catalyst. If desired, this prepolymer can be stored for later processing. The prepolymer can then be heated to approximately 25° C., and mixed for approximately 30 seconds with the 1,4-butanediol chain extender and Bis-(dodecylthio)-dimethylstannane catalyst in a mixing machine using a volumetric ratio of approximately 6.25 prepolymer to 1,4-butanediol chain extender and Bis-(dodecylthio)-dimethylstannane catalyst. After mixing, the contents may be injection into a Teflon coated two part mold heated to approximately 160° C. to cure for approximately 30 minutes. After curing, the mold may be opened and the test strips removed. The implant may be packaged, in packaging such as aluminum foil, and a vacuum may be applied to reduce contact between the implant and moisture in the air. The test pieces were then placed in a dry oven for around 24 hours for annealing and post curing. The test pieces were then sterilized and packaged for use.

The resultant polymer test pieces (strips) were implanted in the coronary arteries of healthy pigs using conventional humane procedures. The coronary arteries were harvested after 30 days and the stented segments of the arteries were fixed and subjected to normal histopathology and histomorphometry studies.

Histopathology of Coronary Arteries

In micrographs from the pig study the stent strut and polymer strip cross-sectional profiles, or the voids created by their loss during sectioning, were present in all histological sections. There tended to be greater vessel injury and inflammation as well as more neointima formation, on the hemicircumference bearing the polymer strip compared to the opposite side, in all sections. An extensive adventitial and perivascular fibrosis with florid neovascularization was also sometimes present on the polymer-bearing side, particularly in the 6235 formulation.

The tunica media was sometimes breached completely by the stent-polymer device on the side adjacent to the polymer, where it appeared to have been disrupted in most cases of breaching by the extensive polymer-associated inflammatory reaction. Again, this was most prominent with the polyurethane formulation.

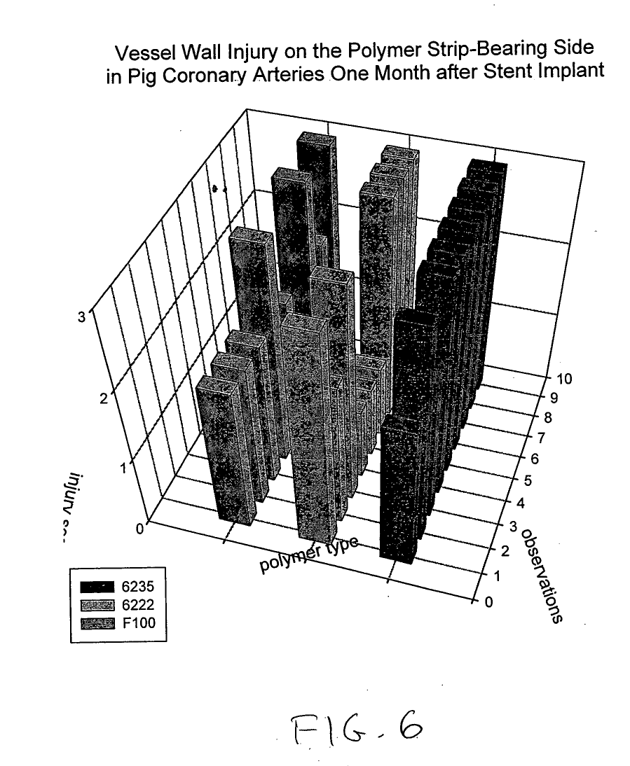

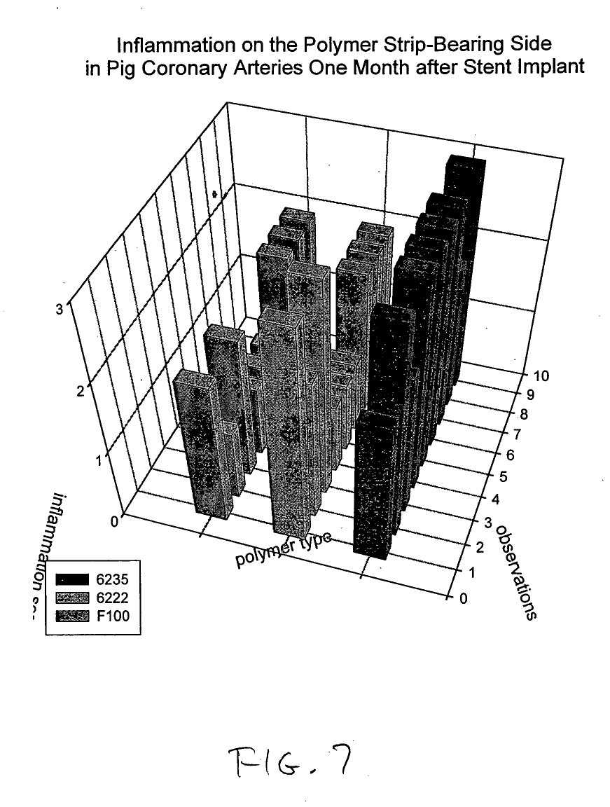

There were no significant effects of polymer type on any of the nominal semi quantitative scoring data, except for two variables. This was an effect of polymer type on neointima score on the polymer-bearing side (H=16.056, P<0.001) with the 6235 polymer showing significantly higher neointima score than both 6222 and F100 polymer. Similarly, inflammation score on the polymer-bearing side showed an effect of polymer type (H=10.249, P=0.006) with the 6235 polymer having a higher median score than the F100 polymer. Graphs depicting the scoring data area are presented as figures (see FIG. 5-7)

There were no significant effects of polymer type on any of the continuous, measured variables of lumen area, neointima area, or neointima thickness.

Conclusions

Experiment now suggests that a successful polyurethane stent will have a polyether soft segment component selected from the group comprising: polytetramethyleneoxide (PTMO) MW 250-2800 Daltons, aliphatic polycarbonate based olygomers MW 600-5000 Daltons, hydroxyl-terminated or amino-terminated olygomers with linear or branched aliphatic backbone such as polyisoprene, polybutadiene, polyisobutylene, carbinol terminated polydimethylsiloxanes (PDMS) or combination of the above-mentioned components.

The hard to soft ratio will be used to tailor the mechanical properties of the device or coating and that chain extenders will include iodine or bromine atoms to improve radiopacity.

The polymer will be injected molded to a near final form for encapsulated and monolithic stents. A substantial annealing process will be required.

The drug loaded into the polymer stent will be selected from the group of antimetabolites of which Rapamycin and Taxol are representative. These drugs are soluble in a variety of solvents and the precise mechanism for inclusion or loading the polymer will need to be the result of additional experiment. At present the applicant is experimenting with a process where the polymer is soaked in an organic solvent to achieve a degree of swelling. Next the polymer is placed in another solvent bath with a high concentration of the drug. An exchange reaction takes place moving the drug into the polymer as the initial solvent is extracted. Experiments with dyes of appropriate molecular weight suggest that this process with be successful for loading drug into the polymer.

Although the examples are intended to be non limiting, the unusual performance of closely related polymers suggest that the specific example is the specie of an as yet undetermined generic invention.

1. A stent device comprising:
a substantially tubular body having a plurality of apertures thereby forming a mesh shape;
said tubular body made from a polyurethane forming a tubular polyurethane body having a soft segment composition and molecular weight, selected from the group consisting of:
polytetramethyleneoxide (PTMO) MW 250-2800 Daltons; aliphatic polycarbonate based olygomers MW 600-5000 Daltons; hydroxyl-terminated or amino-terminated olygomers with linear or branched aliphatic backbone structure typified by polyisoprene, polybutadiene, polyisobutylene, carbinol terminated polydimethylsiloxanes (PDMS);
said tubular polyurethane body storing a drug selected from the group consisting of:
Taxol or Rapamycin.

2. A stent device comprising:
a substantially tubular body having a plurality of apertures thereby forming a mesh shape;
said tubular body made from a metal;
said metal tubular body encapsulated by injection molding with a polyurethane having a soft segment composition and molecular weight, selected from the group consisting of:
polytetramethyleneoxide (PTMO) MW 250-2800 Daltons; aliphatic polycarbonate based olygomers MW 600-5000 Daltons; hydroxyl-terminated or amino-terminated olygomers with linear or branched aliphatic backbone structure typified by polyisoprene, polybutadiene, polyisobutylene, carbinol terminated polydimethylsiloxanes (PDMS), thereby forming a polyurethane encapsulation layer;
said polyurethane encapsulation layer storing a anti metabolite drug.

3. The stent of claim 2 wherein said anti metabolite drug is selected from the group consisting of:
Taxol or Rapamycin.

4. The stent of claim 2 wherein said metal is selected from the group consisting of:
Nitinol or stainless steel.
